(19) United States
(12) Patent Application Publication
(10) Pub. No.: US 2013/0012553 A1
(43) Pub. Date: Jan. 10, 2013

(54) METHODS FOR STABILIZING JOINT DAMAGE IN SUBJECTS USING XANTHINE OXIDOREDUCTASE INHIBITORS

(76) Inventors: Patricia MacDonald, Skokie, IL (US); Lhanoo Gunawardhana, Pleasant Prairie, WI (US)

(21) Appl. No.: 13/521,765

(22) PCT Filed: Feb. 18, 2011
(86) PCT No.: PCT/US11/25450

§ 371 Date: Sep. 26, 2012

Related U.S. Application Data
(60) Provisional application No. 61/306,341, filed on Feb. 19, 2010.

(51) Int. Cl. A61K 31/426; A61P 19/02; G01N 33/48

(52) U.S. Cl. 514/365; 436/99

(57) ABSTRACT

The present invention relates to methods for preventing the progression of joint damage in a subject having hyper-uricemia and who has gout thereof by administering a therapeutically effective amount of at least one xanthine oxidoreductase inhibiting compound or salt thereof. Moreover, the present invention also relates to methods of preventing joint damage in a subject having hyperuricemia and who has gout by administering a therapeutically effective amount of at least one xanthine oxidoreductase inhibiting compound or salt thereof.

### CROSS-REFERENCE TO RELATED APPLICATIONS

This claims priority to U.S. Provisional Patent Application No. 61/306,341, filed on Feb. 19, 2010, the contents of which are hereby incorporated by reference.

### FIELD OF THE INVENTION

In one aspect, the present invention relates to methods of preventing the progression of joint damage in a subject who is hyperuricemic and has gout. In another aspect, the present invention relates to methods of preventing joint damage in a subject who is hyperuricemic and has gout. More specifically, the present invention involves administering to a subject who is hyperuricemic and has gout and in need of prevention of joint damage or prevention of the progression of joint damage a therapeutically effective amount of at least one xanthine oxidoreductase inhibiting compound or salt thereof in order to prevent joint damage or prevent the progression of joint damage in such patients.

### BACKGROUND OF THE INVENTION

Gout affects 3 to 5 million individuals in the United States of America (USA) and is increasing in incidence and prevalence. Gout is a serious health condition characterized by flares of acute arthritis, chronic gouty arthropathy, tophi, and uric acid urolithiasis, and is associated with a broad range of comorbidities, including cardiovascular disease, chronic kidney disease, and metabolic syndrome. At the joint level, a gout flare is best characterized as an acute monoarthritis arthropathy process with proliferative bone reaction that can affect any joint and that can later develop into chronic polyarthritis. Gout attacks tend to occur mostly in the lower extremities and over time additional joints can be involved.

The underlying metabolic aberration in gout is hyperuricemia, which is a condition defined as an elevation in serum urate (sUA) level 6.8 m/dL. Hyperuricemia develops into gout when urate crystals are formed from supersaturated body fluids and deposited in joints, tophi, and parenchymal organs due to a disorder in the urate metabolism. Uric acid is the end product of purine metabolism and is generated in the cascade of hypoxantine→xanthine→uric acid.

Urate-lowering therapy (ULT) is used to treat hyperuricemia in subjects with gout. The goal of ULT is to reduce sUA to 6.0 mg/dL or less, below the concentration at which monosodium urate saturates extracellular fluid. Using ULT to reduce and maintain sUA levels <6.0 mg/dL ultimately improves the clinical symptoms of gout by reducing the frequency of gout flares, decreasing size and number of tophi, and improving quality of life. Due to the potential for paradoxical flares caused by urate crystal mobilization, anti-inflammatory agents and/or colchicine are given as prophylaxis with ULT during the first months of treatment.

Current treatment recommendations focus on initiating ULT in hyperuricemic gout patients who have experienced three (3) or more acute gout flares within the past year. There is no confirmatory evidence addressing the timing of instituting urate-lowering therapy (ULT) earlier in the course of the disease; however, there is no compelling evidence suggesting that the initiation of ULT earlier should not be considered. Data in early gout is currently not available and definitive clinical trials evaluating the timing of joint damage associated with gout have not been completed.

There is some suggestion that joint damage, as a result of crystal deposition, may occur much earlier than previously considered. Monosodium urate crystals (MSU) have been found present in the synovial fluid of joints from asymptomatic hyperuricemic subjects. Their presence would indicate that after the crystals form, they stay within the joint if serum uric acid levels are not reduced to concentrations <6.8 mg/dL. It has been described that about 60% of subjects that experience an initial gout flare will report a second flare within one year and 78% will do so within two years with risk directly associated to serum urate concentrations. The flares, if untreated, may last for three to ten days. Long asymptomatic periods known as “intercritical” are part of the clinical hallmark of the disease.

During the intercritical periods, it is the interaction between the crystals and the cells of the joint that triggers a modest unapparent inflammatory process. Over time, acute intermittent gouty inflammation has been seen to progress to chronic synovitis that may lead to both bone and cartilage destruction. It is known that the innate immune system is critical for the initiation and resolution of monosodium urate (MSU) crystal mediated gouty inflammation. Lowering uric acid levels and maintaining them may reduce acute gout flare episodes, thus halting or reducing the potential future joint damage. There is published evidence of joint damage in patients having frequent or continuous gout flares; however, there is little evidence of when the joint damage begins, and there is no data about the progression of joint damage once it has started.

In gout there is a characteristic proliferative bone reaction to monosodium urate (MSU) crystals that lead to bone destruction. X-rays have been historically used to provide a differential diagnosis based on these characteristic findings. Subcutaneous tophi appear as asymmetric and lobulated soft tissue masses. Presence of intraarticular tophi has been linked to bone erosion. The bone erosion changes observed are well defined with overhanging edges and without associated osteopenia. Compared to rheumatoid arthritis (RA), joint space narrowing appears late in gout.

A variety of imaging techniques are currently in use to evaluate gout. Radiographic evidence is used by the American College of Rheumatology (ACR) as a tool for evaluation of gouty arthritis. Plain radiographs (X-rays) have been successfully used for differentiation between gouty bone erosions present in the form of intraarticular tophi from other forms of erosive arthritis. Radiographic scoring is normally used to analyze the severity and progression of structural damage over time. A frequently used scoring system for X-rays, which was first developed for rheumatoid arthritis, is the modified SHARP scoring (mSS) method which allows manual assessment of erosions and joint space narrowing for joints of both the hands and feet. The modified SHARP score method has been validated in subjects with chronic gout (See Dalbeth et al., “Use of imaging to evaluate gout and other crystal deposition disorders,” Current Opinion in Rheumatology, 21, 124-131 (2009), Dalbeth et al., “Validation of Radiographic Damage Index in Chronic Gout,” Arthritis and Rheumatism, Vol 57, No 6, pp 1067-1073, (Aug. 15, 2007)).

Magnetic Resonance Imaging (MRI) is another technique used that allows for assessment of chronic gout such as sub-clinical erosions, compressive neuropathies, and detection of gouty tophi in atypical locations. MRI has been useful for tophi measurement. It has been determined that the diagnostic utility of plain radiographs and physical examination in tophaceous gout of the hands and wrists underestimated the size and extent of tophaceous involvement of soft tissue and osseous structures when compared to MRI (See Dalbeth, N, et al., “Enhanced Osteoclastogenesis in Patients with Tophaceous Gout”, Arthritis & Rheumatism, 39: 1406-09 (2008), Popp, J D, et al., “Magnetic resonance imaging of tophaceous gout in the hands and wrists”, Semin Arthritis Rheum, 25: 282-9 (1996), herein incorporated by reference. The presence of occult destructive arthropathy has been detected through use of advanced imaging (See Choi M, et al., “Dual Energy Compute Tomography in Tophaceous Gout”, Ann Rheum Dis, 9 Dec. (2008), herein incorporated by reference. This confirmed the limitations of X-rays that other authors have described (See Desiree M F M Van Der Heijde, “Plain X-rays in rheumatoid arthritis: overview of scoring methods, their reliability and applicability”, Bailliere's Clinical Rheumatology, Vol 10, No. 3, August (1996), herein incorporated by reference. A newly developed scoring system for MRI is the OMERACT MRI (RAMRIS) Score in which the presence of bone erosion damage is measured and it is frequently used in the assessment of rheumatoid arthritis. The scoring ranges from 0 to 10, based on the proportion of eroded bone compared with the assessed bone volume judged on all available images, as follows: 0=no erosion; 1=1-10% of bone eroded; 2=11-20% of bone eroded, etc. The OMERACT MRI (RAMRIS) scoring system also includes evaluation of bone marrow edema and synovitis. Another technique that has been successfully used for differentiation of uric acid crystal from calcium crystal deposits in joints is Dual Energy Computed Tomography (DECT). This technique provides valuable information related to varying response of certain tissues to X-rays of different energies. DECT will be used as a complimentary but exploratory tool to compare the results observed with both X-rays and MRI in a preselected group of subjects within this population.

There is a need in the art for a new therapy that can be used to reduce joint damage in hyperuricemic subjects with early gout when assessed through current imaging techniques. Moreover, there is also a need in the art for a new mono therapy that can be used to reduce the progression of joint damage in hyperuricemic subjects with early gout.

### SUMMARY OF THE PRESENT INVENTION

In one embodiment, the present invention relates to a method of preventing the progression of joint damage in a subject who is hyperuricemic and has gout, the method including the steps of:

selecting a patient having at least hyperuricemia and gout; and

administering to the subject who is hyperuricemic and has gout a therapeutically effective amount of a xanthine oxidoreductase inhibitor or a pharmaceutically acceptable salt thereof, wherein the administration of said at least one compound to the subject prevents the progression of joint damage in said subject. More specifically, with respect to the gout, the patient can have early gout or later stage gout.

In another embodiment, the present invention relates to a method of preventing joint damage in a subject who is hyperuricemic and has gout, the method including the step of administering to the subject who is hyperuricemic and has gout a therapeutically effective amount of a xanthine oxidoreductase inhibitor or a pharmaceutically acceptable salt thereof. More specifically, with respect to the gout, the patient can have early gout or later stage gout.

An example of a xanthine oxidoreductase inhibitor that can be used in the modified release dosage forms of methods of the present disclosure are xanthine oxidoreductase inhibitors that comprise the formula:

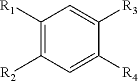

wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;

wherein R₃ and R₄ are each independently a hydrogen or A, B, C or D as shown below:

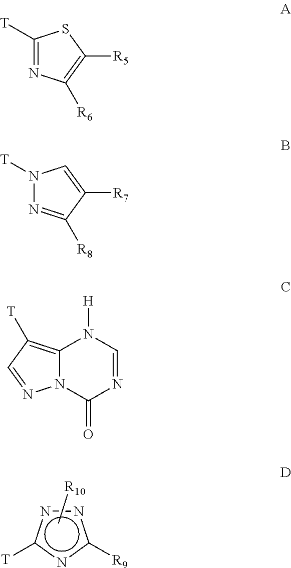

wherein T connects A, B, C or D to the aromatic ring shown above at R₁, R₂, R₃ or R₄.

wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₉ is an unsubstituted pyridyl group or a substituted pyridyl group; and

wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above.

Examples of compounds having the above formula are: (a) 2-[3-cyano-4-(2-methylpropoxy)phenyl]-4-methylthiazole-5-carboxylic acid or a pharmaceutically acceptable salt thereof; (b) 2-[3-cyano-4-(3-hydroxy-2-methylpropoxy)phenyl]-4-methyl-5-thiazolecarboxylic acid or a pharmaceutically acceptable salt thereof; (c) 2-[3-cyano-4-(2-hydroxy-2-methylpropoxy)phenyl]-4-methyl-5-thiazolecarboxylic acid or a pharmaceutically acceptable salt thereof; (d) 2-(3-cyano-4-hydroxyphenyl)-4-methyl-5-thiazolecarboxylic acid or a pharmaceutically acceptable salt thereof; (e) 2-[4-(2-carboxypropoxy)-3-cyanophenyl]-4-methyl-5-thiazolecarboxylic acid or a pharmaceutically acceptable salt thereof; (f) 1-3-cyano-4-(2,2-dimethylpropoxy)phenyl]-1H-pyrazole-4-carboxylic acid or a pharmaceutically acceptable salt thereof. (g) pyrazolo[1,5-a]-1,3,5-triazin-4-(1H)-one, 8-[3-methoxy-4-(phenylsulfinyl)phenyl]-sodium salt (±); and (h) 3-(2-methyl-4-pyridyl)-5-cyano-4-isobutoxyphenyl)-1,2,4-triazole or a pharmaceutically acceptable salt thereof.

Another example of at least one xanthine oxidoreductase inhibitor that can be used in the methods of the present disclosure are xanthine oxidoreductase inhibitors that comprise the formula:

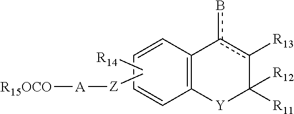

wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;

wherein R₁₃ is a hydrogen or a substituted or unsubstituted lower alkyl group;

wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R_{17′}, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R_{17′} are each independently a hydrogen or a substituted or unsubstituted lower alkyl;

wherein R₁₅ is a hydrogen or a pharmaceutically active ester-forming group;

wherein A is a straight or branched hydrocarbon radical having one to five carbon atoms;

wherein B is a halogen, an oxygen, or a ethylenedithio;

wherein Y is an oxygen, a sulfur, a nitrogen or a substituted nitrogen;

wherein Z is an oxygen, a nitrogen or a substituted nitrogen; and

the dotted line refers to either a single bond, a double bond, or two single bonds.

A subject being treated pursuant to the above described methods of the invention can have one or more of the following conditions: acute gouty arthritis, chronic gouty joint disease, tophaceous gout, and uric acid urolithiasis. In addition, the subject can further exhibit at least one of: inflammation developed within one (1) day, monoarticular arthritis, redness observed over one or more joints, a first metatarsophalangeal joint painful or swollen, an unilateral first metatarsophalangeal joint attack, unilateral tarsal joint attack, tophus (proven or suspected), asymmetric swelling within a joint on X-ray, sub-cortical cysts without erosions (which can be confirmed via X-ray), joint fluid culture negative for organisms during attacks, a tophus proven to contain urate crystals (which can be shown by chemical or polarized light microscopic means), characteristic urate crystals in the joint fluid or combinations thereof. Subjects being treated can have a serum uric acid level of ≧7.0 mg/dL.

### DETAILED DESCRIPTION OF THE INVENTION

### Definitions

As used herein, the singular forms “a,” “an” and “the” include plural referents unless the context clearly dictates otherwise.

The term “about” is used synonymously with the term “approximately.” Illustratively, the use of the term “about” indicates that values slightly outside the cited values, namely, plus or minus 10%. Such dosages are thus encompassed by the scope of the claims reciting the terms “about” and “approximately.”

The terms “administer”, “administering”, “administered” or “administration” refer to any manner of providing a drug (such as, a xanthine oxidoreductase inhibitor or a salt thereof) to a subject or patient. Routes of administration can be accomplished through any means known by those skilled in the art. Such means include, but are not limited to, oral, buccal, intravenous, subcutaneous, intramuscular, by inhalation and the like.

As used herein, the term “gout” refers to a subject that is hyperuricemic and has experienced or has a history of one or more gout flares in a joint during said subject's lifetime. The joint can include, but is not limited to, the right or left metatarsophalangeal joint (MTP), ankle, instep, wrist, shoulder, hip, knee, elbow, metacarpal phalangeal (MCP), interphalangeal (IP), proximal inter-phalangeal (PIP), or distal inter-phalangeal (DIP) joints. There are a number of stages of gout. For example, patients or subjects suffering from or having “early gout” as used herein, are those patients that are hyperuricemic and that have experienced or have a history of only a single gout flare in a joint during said subject's lifetime. Patients or subjects suffering from or having “later stage gout” as used herein, are those patients that arehyperuricemic and that have experienced or have a history of more than one (or multiple) gout flares in a joint during said subject's lifetime. The term “gout” as used herein refers to both “early gout” and “later stage gout” collectively.

As used herein, the term “pharmaceutically acceptable” includes moieties or compounds that are, within the scope of sound medical judgment, suitable for use in contact with the tissues of humans and lower animals without undue toxicity, irritation, allergic response, and the like, and are commensurate with a reasonable benefit/risk ratio.

As used herein, the term “subject” refers to an animal, preferably a mammal, including a human or non-human. The terms patient and subject may be used interchangeably herein.

The terms “therapeutically effective amount” or “prophylactically effective amount” of a drug (namely, at least one xanthine oxidoreductase inhibitor or a salt thereof) refers to a nontoxic but sufficient amount of the drug to provide the desired effect of maintaining or preserving joint integrity in a subject. In other words, these terms mean a sufficient amount of, for example, the composition, xanthine oxidoreductase inhibiting compound, or formulation necessary to preserve the subject's joint integrity at a reasonable benefit/risk ratio applicable to any medical treatment. As used herein, the term “joint integrity” refers to the stabilization of a joint when a comparison is made to the same joint at baseline. The baseline for determining such joint integrity can be established using routine techniques in the art, such as, but not limited to, Magnetic Resonance Imaging (MRI), plain radiographs (X-rays), and Dual Energy Computed Tomography (DECT). The baseline can be original or previously or last obtained MRI, X-ray or DECT of the joint being examined (such as the last X-ray obtained during a visit to a physician's office). Stabilization can be determined by obtaining a MRI, X-ray or DECT of the joint being examined at a current time point and then comparing one or more of inflammation, bone destruction, bone erosion, joint space narrowing, bone marrow edema, synovitis, cartilage destruction or combinations thereof evidenced in said MRI, X-ray or DECT with one or more one or more of inflammation, bone destruction, bone erosion, joint space narrowing, bone marrow edema, synovitis, cartilage destruction or combinations thereof contained in the original or previously obtained MRI, X-ray or DECT (namely, the baseline). A determination of joint integrity is then made based on said comparison. As with other pharmaceuticals, it will be understood that the total daily usage of a pharmaceutical composition of the invention will be decided by a patient's attending physician within the scope of sound medical judgment. The specific therapeutically effective or prophylactically effective dose level for any particular patient will depend upon a variety of factors including the disorder being treated and the severity of the disorder; activity of the specific compound employed; the specific composition employed; the age, body weight, general health, sex and diet of the patient; the time administration, route of administration, and rate of excretion of the specific compound employed; the duration of the treatment; drugs used in combination or coincidental with the specific compound employed; and other factors known to those of ordinary skill in the medical arts. For example, it is well within the skill of the art to start doses of the compound at levels lower than required to achieve the desired therapeutic effect and to gradually increase the dosage until the desired effect is achieved.

Accordingly, the amount of drug that is “effective” or “prophylactic” will vary from subject to subject, depending on the age and general condition of the individual, the particular drug or drugs, and the like. Thus, it is not always possible to specify an exact “therapeutically effective amount” or a “prophylactically effective amount”. However, an appropriate “therapeutically effective amount” or “prophylactically effective amount” in any individual case may be determined by one skilled in the art.

The term “dosage form” refers to any solid object, semi-solid, or liquid composition designed to contain a specific pre-determined amount (i.e., dose) of a certain active agent. Suitable dosage forms may be pharmaceutical drug delivery systems, including those for oral administration, buccal administration, rectal administration, topical or mucosal delivery or subcutaneous implants, or other implanted drug delivery systems and the like. Preferably, the dosage forms of the present disclosure are considered to be solid, however, they may contain liquid or semi-solid components. More preferably, the dosage form is an orally administered system for delivering an active agent to the gastrointestinal tract of a subject.

The terms “treating” and “treatment” refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease.

As used herein, the term, “test sample” generally refers to a biological material being tested for and/or suspected of containing an analyte of interest, such as serum urate. The biological material may be derived from any biological source but preferably is a biological fluid likely to contain serum urate. Examples of biological materials include, but are not limited to, whole blood, serum, plasma, red blood cells, platelets, etc. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc.

As used herein, the term “xanthine oxidoreductase inhibitor” refers to any compound that (1) is an inhibitor of a xanthine oxidoreductase, such as, but not limited to, xanthine oxidase; and (2) chemically, does not contain a purine ring in its structure (i.e. is a “non-purine”). The phrase “xanthine oxidoreductase inhibitor” as defined herein also includes metabolites, polymorphs, solvates and prodrugs of the such compounds, including metabolites, polymorphs, solvates and prodrugs of the exemplary compounds described as Formula I and Formula II below. Examples of xanthine oxidoreductase inhibitors include, but are not limited to, 2-[4-(2-carboxypropoxy)-3-cyanophenyl]-4-methyl-5-thiazolecarboxylic acid and compounds having the following Formula I or Formula II:

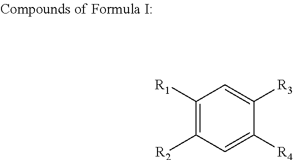

wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;

wherein R₃ and R₄ are each independently a hydrogen or A, B, C or D as shown below:

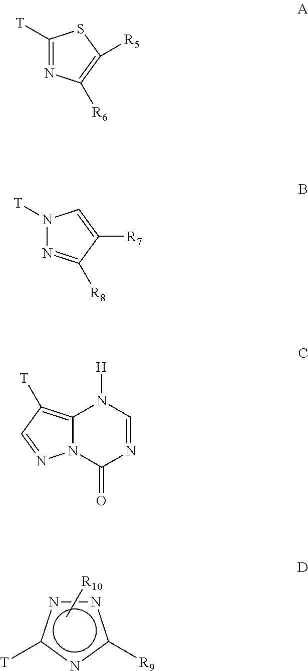

wherein T connects or attaches A, B, C or D to the aromatic ring shown above at R₁, R₂, R₃ or R₄.

wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;

wherein R₉ is an unsubstituted pyridyl group or a substituted pyridyl group; and

wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above in Formula I.

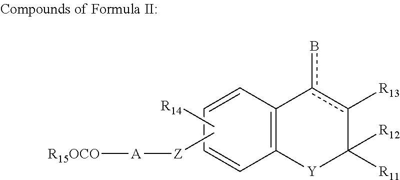

wherein R₁₁ and R₁₂ are each independently a hydrogen, a substituted or unsubstituted lower alkyl group, a substituted or unsubstituted phenyl (the substituted phenyl in this Formula II refers to a phenyl substituted with a halogen or lower alkyl, and the like. Examples include, but are not limited to, p-tolyl and p-chlorophenyl), or R₁₁ and R₁₂ may together form a four- to eight-membered carbon ring together with the carbon atom to which they are attached;

wherein R₁₃ is a hydrogen or a substituted or unsubstituted lower alkyl group;

wherein R₁₄ is one or two radicals selected from a group consisting of a hydrogen, a halogen, a nitro group, a substituted or unsubstituted lower alkyl group, a substituted or unsubstituted phenyl (the substituted phenyl in this Formula II refers to a phenyl substituted with a halogen or lower alkyl group, and the like. Examples include, but are not limited to, p-tolyl and p-chlorophenyl), —OR₁₆ and —SO₂NR₁₇R_{17′}, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R_{17′} are each independently a hydrogen or a substituted or unsubstituted lower alkyl group;

wherein R₁₅ is a hydrogen or a pharmaceutically active ester-forming group;

wherein A is a straight or branched hydrocarbon radical having one to five carbon atoms;

wherein B is a halogen, an oxygen, or a ethylenedithio;

wherein Y is an oxygen, a sulfur, a nitrogen or a substituted nitrogen;

wherein Z is an oxygen, a nitrogen or a substituted nitrogen; and

the dotted line refers to either a single bond, a double bond, or two single bonds (for example, when B is ethylenedithio, the dotted line shown in the ring structure can be two single bonds).

As used herein, the term “lower alkyl(s)” group refers to a C₁-C₇ alkyl group, including, but not limited to, including methyl, ethyl, n-propyl, isopropyl, butyl, isobutyl, sec-butyl, tert-butyl, pentyl, isopentyl, hexyl, heptal and the like.

As used herein, the term “lower alkoxy” refers to those groups formed by the bonding of a lower alkyl group to an oxygen atom, including, but not limited to, methoxy, ethoxy, propoxy, isopropoxy, butoxy, isobutoxy, pentoxy, hexoxy, heptoxy and the like.

As used herein, the term “lower alkylthio group” refers to those groups formed by the bonding of a lower alkyl to a sulfur atom.

As used herein, the term “halogen” refers to fluorine, chlorine, bromine and iodine.

As used herein, the term “substituted pyridyl” refers to a pyridyl group that can be substituted with a halogen, a cyano group, a lower alkyl, a lower alkoxy or a lower alkylthio group.

As used herein, the term “four- to eight-membered carbon ring” refers to cyclobutyl, cyclopentyl, cyclohexyl, cycloheptyl, cyclooctyl and the like.

As used herein, the phrase “pharmaceutically active ester-forming group” refers to a group that binds to a carboxyl group through an ester bond. Such ester-forming groups can be selected from carboxy-protecting groups commonly used for the preparation of pharmaceutically active substances, especially prodrugs. For the purpose of the invention, said group should be selected from those capable of binding to compounds having Formula II wherein R₁₅ is hydrogen through an ester bond. Resultant esters are effective to increase the stability, solubility, and absorption in gastrointestinal tract of the corresponding non-esterified forms of said compounds having Formula II, and also prolong the effective blood-level of it. Additionally, the ester bond can be cleaved easily at the pH of body fluid or by enzymatic actions in vivo to provide a biologically active form of the compound having Formula II. Preferred pharmaceutically active ester-forming groups include, but are not limited to, 1-(oxygen substituted)-C₂ to C₁₅ alkyl groups, for example, a straight, branched, ringed, or partially ringed alkanoyloxyalkyl groups, such as acetoxymethyl, acetoxyethyl, propionyloxymethyl, pivaloyloxymethyl, pivaloyloxyethyl, cyclohexaneacetoxyethyl, cyclohexanecarbonyloxycyclohexylmethyl, and the like, C₃ to C₁₅ alkoxycarbonyloxyalkyl groups, such as ethoxycarbonyloxyethyl, isopropoxycarbonyloxyethyl, isopropoxycarbonyloxypropyl, t-butoxycarbonyloxyethyl, isopentyloxycarbonyloxypropyl, cyclohexyloxycarbonyloxyethyl, cyclohexylmethoxycarbonyloxyethyl, bornyloxycarbonyloxyisopropyl, and the like, C₂ to C₈ alkoxyalkyls, such as methoxy methyl, methoxy ethyl, and the like, C₄ to C₈ 2-oxacycloalkyls such as, tetrahydropyranyl, tetrahydrofuranyl, and the like, substituted C₈ to C₁₂ aralkyls, for example, phenacyl, phthalidyl, and the like, C₆ to C₁₂ aryl, for example, phenyl xylyl, indanyl, and the like, C₂ to C₁₂ alkenyl, for example, allyl, (2-oxo-1,3-dioxolyl)methyl, and the like, and [4,5-dihydro-4-oxo-1H-pyrazolo[3,4-d]pyrimidin-1-yl]methyl, and the like.

In R₁₆ in Formula II, the term “ester” as used in the phrase “the ester of carboxymethyl” refers to a lower alkyl ester, such as methyl or ethyl ester; and the term “ether” used in the phrase “the ether of hydroxyethyl” means an ether which is formed by substitution of the hydrogen atom of hydroxyl group in the hydroxyethyl group by aliphatic or aromatic alkyl group, such as benzyl.

The carboxy-protecting groups may be substituted in various ways. Examples of substituents include halogen atom, alkyl groups, alkoxy groups, alkylthio groups and carboxy groups.

As used herein, the term “straight or branched hydrocarbon radical” in the definition of A in Formula II above refers to methylene, ethylene, propylene, methylmethylene, or isopropylene.

As used herein, the substituent of the “substituted nitrogen” in the definition of Y and Z in Formula II above are hydrogen, lower alkyl, or acyl.

As used herein, the term “phenyl-substituted lower alkyl” refers to a lower alkyl group substituted with phenyl, such as benzyl, phenethyl or phenylpropyl.

As used herein, the term “prodrug” refers to a derivative of the compounds shown in the above-described Formula I and Formula II that have chemically or metabolically cleavable groups and become by solvolysis or under physiological conditions compounds that are pharmaceutically active in vivo. Esters of carboxylic acids are an example of a prodrug that can be used in the dosage forms of the present invention. Methyl ester prodrugs may be prepared by reaction of a compound having the above-described formula in a medium such as methanol with an acid or base esterification catalyst (e.g., NaOH, H₂SO₄). Ethyl ester prodrugs are prepared in similar fashion using ethanol in place of methanol.

Examples of compounds having the above Formula I are: 2-[3-cyano-4-(2-methylpropoxy)phenyl]-4-methylthiazole-5-carboxylic acid (also known as “febuxostat”), 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-Cyano-4-(2,2-dimethylpropoxy)phenyl]-1H-pyrazole-4-carboxylic acid, pyrazolo[1,5-a]-1,3,5-triazin-4-(1H)-one, 8-[3-methoxy-4-(phenylsulfinyl)phenyl]-sodium salt (±) or 3-(2-methyl-4-pyridyl)-5-cyano-4-isobutoxyphenyl)-1,2,4-triazole.

Preferred compounds having the above Formula I are: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid. These preferred compounds have also been found not have an effect at a therapeutically effective amount in a subject on the activity of any of the following enzymes involved in purine and pyrimidine metabolism: guanine deaminase, hypoxanthine-guanine phosphoribosyltransferse, purine nucleotide phosphorylase, orotate phosphoribosyltransferase or orotidine-5-monophosphate decarboxylase (i.e., meaning that it is “selective” for none of these enzymes which are involved in purine and pyrimidine metabolism). Assays for determining the activity for each of the above-described enzymes is described in Yasuhiro Takano, et al., Life Sciences, 76:1835-1847 (2005). These preferred compounds have also been referred to in the literature as nonpurine, selective inhibitors of xathine oxidase (NP/SIXO).

Examples of compounds having the above Formula II are described in U.S. Pat. No. 5,268,386 and EP 0 415 566 A1.

With the exception of pyrazolo[1,5-a]-1,3,5-triazin-4-(1H)-one, 8-[3-methoxy-4-(phenylsulfinyl)phenyl]-sodium salt (±), methods for making xanthine oxidoreductase inhibiting compounds of Formulas I and II for use in the methods of the present invention are known in the art and are described, for example, in U.S. Pat. Nos. 5,268,386, 5,614,520, 6,225,474, 7,074,816 and EP 0 415 566 A1 and in the publications Ishibuchi, S. et al., Bioorg. Med. Chem. Lett., 11:879-882 (2001) and which are each herein incorporated by reference. Other xanthine oxidoreductase inhibiting compounds can be found using xanthine oxidoreductase and xanthine in assays to determine if such candidate compounds inhibit conversion of xanthine into uric acid. Such assays are well known in the art.

Pyrazolo[1,5-a]-1,3,5-triazin-4-(1H)-one, 8-[3-methoxy-4-(phenylsulfinyl)phenyl]-sodium salt (±) is available from Otsuka Pharmaceutical Co. Ltd. (Tokyo, Japan) and is described in the following publications: Uematsu T., et al., “Pharmacokinetic and Pharmacodynamic Properties of a Novel Xanthine Oxidase Inhibitor, BOF-4272, in Healthy Volunteers, J. Pharmacology and Experimental Therapeutics, 270:453-459 (August 1994), Sato, S., A Novel Xanthine Deydrogenase Inhibitor (BOF-4272). In Purine and Pyrimidine Metabolism in Man, Vol. VII, Part A, ed. By P. A. Harkness, pp. 135-138, Plenum Press, New York. Pyrazolo[1,5-a]-1,3,5-triazin-4-(1H)-one, 8-[3-methoxy-4-(phenylsulfinyl)phenyl]-sodium salt (±) can be made using routine techniques known in the art.

### DESCRIPTION OF THE INVENTION

2-[3-cyano-4-(2-methylpropoxy)phenyl]-4-methylthiazole-5-carboxylic acid (also known as “febuxostat” and “TMX-67”) is a potent, nonpurine selective inhibitor of xanthine oxidoreductase that exhibits antihyperuricemic activity by reducing the formation of uric acid in patients with gout. Febuxostat is a potent selective inhibitor of the xanthine oxidoreductase enzyme (or xanthine oxidoreductase inhibitor) that is required for the synthesis of uric acid.

In one embodiment, the present invention relates to methods of preventing the progression of joint damage in subjects in need thereof. In a second embodiment, the present invention relates to methods of preventing joint damage in a subject of need thereof. It has been discovered that a class of compounds known as xanthine oxidoreductase inhibitors can be used not only to reduce serum uric acid levels in subjects, but also to (1) prevent the progression of joint damage in said subjects over time; and (2) prevent joint damage in said subjects over time. In a third embodiment, the present invention relates to methods of preventing joint damage in a subject of need thereof. It has been discovered that urate lowering compounds can be used not only to reduce serum uric acid levels in subjects, but also to (1) prevent the progression of joint damage in said subjects over time; and (2) prevent joint damage in said subjects over time. As used herein, urate lowering compounds are compounds, other than xanthine oxidoreductase inhibitors, which are capable of reducing serum uric acid levels (or serum urate levels) in a subject. Examples of urate lowering compounds include, but are not limited to, xanthine oxidase inhibitors (such as, but not limited to allopurinol, oxypurinol, etc) and uricosurics (such as, but not limited to, probenecid, benzbromarone and sulfinpyrazone, etc.)

Because the xanthine oxidoreductase inhibitors of the present invention are effective in reducing the formation of uric acid, these compounds can be used to treat subjects suffering from or having hyperuricemia, gout, acute gouty arthritis, chronic gouty disease, tophaceous gout, uric acid urolithiasis. More specifically, the subject selected for treatment pursuant to the methods of the present invention is at least hyperuricemic and has gout. Specifically, the subject is suffering from or having early gout. Alternatively, the subject is suffering from or having later stage gout. The gout flare(s) is located within a joint, such as, but not limited to, the right or left Metatarsophalangeal joint (MTP), ankle, instep, wrist, shoulder, hip, knee, elbow, metacarpal phalangeal (MCP), interphalangeal (IP), proximal inter-phalangeal (PIP), or distal inter-phalangeal (DIP) joints. If multiple gout flares have been experienced, they can occur in the same joint or in different joints. In addition to being hyperuricemic and having gout, the subject an may also exhibit or have a history of one or more of the following: inflammation developed within one (1) day, monoarticular arthritis, redness observed over one or more joints, a first metatarsophalangeal joint painful or swollen, an unilateral first metatarsophalangeal joint attack, unilateral tarsal joint attack, tophus (proven or suspected), asymmetric swelling within a joint on X-ray, sub-cortical cysts without erosions (which can be confirmed via X-ray), joint fluid culture negative for organisms during attacks, a tophus proven to contain urate crystals (which can be shown by chemical or polarized light microscopic means), characteristic urate crystals in the joint fluid or combinations thereof. Such treatments involve the administration of sufficient amounts of xanthine oxidoreductase inhibitor to reduce serum uric acid levels in the subject with a quick onset (namely, within one week of first beginning treatment with a xanthine oxidoreductase inhibitor (See, Becker M, Kisicki J, Khosravan R, Wu J, Mulford D, Hunt B, MacDonald P, Joseph-Ridge N., Nucleosides Nucleotides Nucleic Acids, 23(8 & 9):1111-1116 (October 2004)) and maintain a reduction in the subject's serum urate level for a prolonged period, such as for at least 4 weeks (See, Becker M A, Schumacher H R Jr, Wortmann R L, MacDonald P A, Palo W A, Eustace D, Vernillet L, Joseph-Ridge N, Arthritis Rheum., 52(3):916-923 (March 2005)), at least a year, at least two years, at least 30 months (See, Becker M A, Schumacher H R Jr, Wortmann R L, MacDonald P A, Eustace D, Palo W A, Streit J, Joseph-Ridge N., N Engl J. Med., 354(6):1532-1533 (April 2006)), at least 36 months, at least 42 months, at least 48 months, at least 54 months, at least 60 months, at least 66 months, at least 72 months, at least 78 months, at least 84 months, at least 90 months, at least 96 months, at least 102 months, at least 108 months, at least 114 months, at least 120 months and beyond.

It was discovered that administering xanthine oxidoreductase inhibitors in quantities that are effective to reduce a subject's serum uric acid level (or serum urate level) for such prolonged periods is also therapeutically effective in preventing the progression of the subject's joint damage during such periods. The prevention of the progression of a subject's joint damage can be assessed by well-known measures, such as, but not limited to, Magnetic Resonance Imaging (MRI), plain radiographs (X-rays), and Dual Energy Computed Tomography (DECT). It will be understood that preventing the progression of a subject's joint damage entails not only maintaining the joint in xanthine oxidoreductase inhibitor-treated subjects when compared to placebo-treated subjects, but also maintaining the joint reasonably close to baseline levels, i.e., at stable levels, or in other words, stabilizing the joint from one or more of further inflammation, bone destruction, bone erosion, joint space narrowing, bone marrow edema, synovitis, cartilage destruction or combinations thereof in xanthine oxidoreductase inhibitor-treated subjects when compared to placebo-treated subjects. Moreover, it was further discovered that administering xanthine oxidoreductase inhibitors in quantities that are effective to reduce a subject's serum uric acid level for such prolonged periods is also therapeutically effective in preventing joint damage in the subject during such periods.

As alluded to above, following the administration of the xanthine oxidoreductase inhibitors of the present invention, the progression of joint damage or bone erosion in a subject is assessed by a variety of imaging techniques, such as Magnetic Resonance Imaging (MRI), plain radiographs (X-rays), and Dual Energy Computed Tomography (DECT). If a MRI is used, any scoring system, such as, the OMERACT MRI (RAMRIS) scoring system can be used. With RAMRIS, the presence of bone erosion damage is measured and it is frequently used in the assessment of rheumatoid arthritis. The scoring ranges from 0 to 10, based on the proportion of eroded bone compared with the assessed bone volume judged on all available images, as follows: 0=no erosion; 1=1-10% of bone eroded; 2=11-20% of bone eroded, etc. The OMERACT MRI (RAMRIS) scoring system also includes evaluation of bone marrow edema and synovitis.

It has been found that the administration of the xanthine oxidoreductase inhibitors of the present invention can also be used to prevent joint damage in subjects. Such subjects may or may not be suffering from or having acute gouty arthritis, chronic gouty disease, tophaceous gout, uric acid urolithiasis. Preferred subjects for the prevention of further joint damage are those individuals suffering from or having hyperuricemia and who have gout. Specifically, the subjects have early gout. Alternatively, the subject has later stage gout. In general, subjects particularly suitable for treatment according to the methods of the present invention are those individuals having serum urate levels greater than about 6.0 mg/dL, greater than about 7.0 mg/dL, greater than about 8.0 mg/dL, greater than about 9.0 mg/dL, greater than about 10.0 mg/dL, or greater than about 11.0 mg/dL. For subject's having a serum urate level from about 6.0 mg/dL, greater than about 7.0 mg/dL, greater than about 8.0 mg/dL, greater than about 9.0 mg/dL, greater than about 10.0 mg/dL, or greater than about 11.0 mg/dL, any reduction in serum urate resulting from the administration of at least one xanthine oxidoreductase inhibitor according to the methods of the present invention can be used to prevent the progression joint damage in a patient who has previously suffered joint damage (such as, for example, the result of the deposition of monosodium urate crystals resulting from hyperuricemia) over time. Alternatively, for subject's having a serum urate level from about 6.0 mg/dL, greater than about 7.0 mg/dL, greater than about 8.0 mg/dL, greater than about 9.0 mg/dL, greater than about 10.0 mg/dL, or greater than about 11.0 mg/dL., any reduction in serum urate resulting from the administration of at least one xanthine oxidoreductase inhibitor according to the methods of the present invention can be used to prevent joint damage over time in a patient who has not previously suffered joint damage. The treatment of subjects involves the administration of therapeutically effective amount of xanthine oxidoreductase inhibitor to prevent joint damage from occurring in a subject and maintaining such prevention of joint damage, or in the cases of those subjects who have previously suffered joint damage, the progression of any further joint damage, for a prolonged period, such as at least 4 weeks, at least a year, at least two years, at least 30 months, at least 36 months, at least 42 months, at least 48 months, at least 54 months, at least 60 months, at least 66 months, at least 72 months, at least 78 months, at least 84 months, at least 90 months, at least 96 months, at least 102 months, at least 108 months, at least 114 months, at least 120 months and beyond.

The present invention also relates to diagnostic methods for identifying a subject or patient suitable for treatment. More specifically, the diagnostic methods of the present invention involve identifying a patient or subject suitable for treatment with at least one xanthine oxidoreductase inhibitor, such as, for example, to prevent joint damage or the progression of joint damage in a subject. The first step of such a method involves obtaining a test sample from a subject or patient. The test sample can be collected using routine techniques known in the art. Once such a test sample is obtained, the serum urate levels or serum uric acid levels of said patient or subject is determined. Specifically, a determination is made whether or not the subject is hyperuricemic (namely, the subject has a serum urate or serum uric acid level greater than or equal to 7.0 mg/dL). If the subject is determined to be or is classified as being hyperuricemic, then a further determination is made whether or not the patient also suffers from gout, such as, for example, early gout or later stage gout. If the hyperuricemic subject is also identified as having early or later stage gout, then the subject is determined to be eligible for treatment with at least one xanthine oxidoreductase inhibitor in order to prevent joint damage or the progression of joint damage in said subject. However, if the hyperuricemic subject is not identified as having early or later stage gout, then the subject is determined not to be eligible for treatment with at least one xanthine oxidoreductase inhibitor in order to prevent joint damage or the progression of joint damage in said subject.

In another embodiment, the present invention relates to administering to a subject in need thereof a therapeutically effective amount at least one urate lowering compound (xanthine oxidase inhibitors (such as, but not limited to allopurinol, oxypurinol, etc) and uricosurics (such as, but not limited to, probenecid, benzbromarone and sulfinpyrazone, etc.)) to reduce a subject's serum uric acid level (or serum urate level) for a prolonged period (said prolonged period being those disclosed above in connection with the at least one xanthine oxidoreductase inhibitor) to prevent the progression of the subject's joint damage during such periods. As described above, the prevention of the progression of a subject's joint damage can be assessed by well-known measures, such as, but not limited to, Magnetic Resonance Imaging (MRI), plain radiographs (X-rays), and Dual Energy Computed Tomography (DECT). It will be understood that preventing the progression of a subject's joint damage entails not only maintaining the joint in urate lowering compound-treated subjects when compared to placebo-treated subjects, but also maintaining the joint reasonably close to baseline levels, i.e., at stable levels, or in other words, stabilizing the joint from one or more of further inflammation, bone destruction, bone erosion, joint space narrowing, bone marrow edema, synovitis, cartilage destruction or combinations thereof in urate lowering compound-treated subjects when compared to placebo-treated subjects. Moreover, administering xanthine oxidoreductase inhibitors in quantities that are effective to reduce a subject's serum uric acid level for the above prolonged periods may also therapeutically effective in preventing joint damage in the subject during such periods described above (namely, for at least 4 weeks at least a year, at least two years, at least 30 months at least 36 months, at least 42 months, at least 48 months, at least 54 months, at least 60 months, at least 66 months, at least 72 months, at least 78 months, at least 84 months, at least 90 months, at least 96 months, at least 102 months, at least 108 months, at least 114 months, at least 120 months and beyond).

As alluded to above, following the administration of the urate lowering compound, the progression of joint damage or bone erosion in a subject is assessed by a variety of imaging techniques, such as Magnetic Resonance Imaging (MRI), plain radiographs (X-rays), and Dual Energy Computed Tomography (DECT). If a MRI is used, any scoring system, such as, the OMERACT MRI (RAMRIS) scoring system can be used. With RAMRIS, the presence of bone erosion damage is measured and it is frequently used in the assessment of rheumatoid arthritis. The scoring ranges from 0 to 10, based on the proportion of eroded bone compared with the assessed bone volume judged on all available images, as follows: 0=no erosion; 1=1-10% of bone eroded; 2=11-20% of bone eroded, etc. The OMERACT MRI (RAMRIS) scoring system also includes evaluation of bone marrow edema and synovitis.

The administration of the urate lowering compound can also be used to prevent joint damage in subjects. Such subjects may or may not be suffering from or having acute gouty arthritis, chronic gouty disease, tophaceous gout, uric acid urolithiasis. Preferred subjects for the prevention of further joint damage are those individuals suffering from or having hyperuricemia and who have gout. Specifically, the subjects have early gout. Alternatively, the subject has later stage gout. In general, subjects particularly suitable for treatment according to the methods of the present invention are those individuals having serum urate levels greater than about 6.0 mg/dL, greater than about 7.0 mg/dL, greater than about 8.0 mg/dL, greater than about 9.0 mg/dL, greater than about 10.0 mg/dL, or greater than about 11.0 mg/dL. For subject's having a serum urate level from about 6.0 mg/dL, greater than about 7.0 mg/dL, greater than about 8.0 mg/dL, greater than about 9.0 mg/dL, greater than about 10.0 mg/dL, or greater than about 11.0 mg/dL, any reduction in serum urate resulting from the administration of at least one urate lowering compound according to the methods of the present invention can be used to prevent the progression joint damage in a patient who has previously suffered joint damage (such as, for example, the result of the deposition of monosodium urate crystals resulting from hyperuricemia) over time. Alternatively, for subject's having a serum urate level from about 6.0 mg/dL, greater than about 7.0 mg/dL, greater than about 8.0 mg/dL, greater than about 9.0 mg/dL, greater than about 10.0 mg/dL, or greater than about 11.0 mg/dL., any reduction in serum urate resulting from the administration of at least one urate lowering compound according to the methods of the present invention can be used to prevent joint damage over time in a patient who has not previously suffered joint damage. The treatment of subjects involves the administration of therapeutically effective amount of urate lowering compound to prevent joint damage from occurring in a subject and maintaining such prevention of joint damage, or in the cases of those subjects who have previously suffered joint damage, the progression of any further joint damage, for a prolonged period, such as at least 4 weeks, at least a year, at least two years, at least 30 months, at least 36 months, at least 42 months, at least 48 months, at least 54 months, at least 60 months, at least 66 months, at least 72 months, at least 78 months, at least 84 months, at least 90 months, at least 96 months, at least 102 months, at least 108 months, at least 114 months, at least 120 months and beyond.

The present invention also relates to diagnostic methods for identifying a subject or patient suitable for treatment. More specifically, the diagnostic methods of the present invention involve identifying a patient or subject suitable for treatment with at least one urate lowering compound, such as, for example, to prevent joint damage or the progression of joint damage in a subject. The first step of such a method involves obtaining a test sample from a subject or patient. The test sample can be collected using routine techniques known in the art. Once such a test sample is obtained, the serum urate levels or serum uric acid levels of said patient or subject is determined. Specifically, a determination is made whether or not the subject is hyperuricemic (namely, the subject has a serum urate or serum uric acid level greater than or equal to 7.0 mg/dL). If the subject is determined to be or is classified as being hyperuricemic, then a further determination is made whether or not the patient also suffers from gout, such as, for example, early gout or later stage gout. If the hyperuricemic subject is also identified as having early or later stage gout, then the subject is determined to be eligible for treatment with at least one urate lowering compound in order to prevent joint damage or the progression of joint damage in said subject. However, if the hyperuricemic subject is not identified as having early or later stage gout, then the subject is determined not to be eligible for treatment with at least urate lowering compound in order to prevent joint damage or the progression of joint damage in said subject. Compositions containing at least one xanthine oxidoreductase inhibitor, at least one urate lowering compound (such as at least one xanthine oxidase inhibitor or at least one uricosuric) or combinations thereof are contemplated for use in the methods of the present invention. Using the excipients and dosage forms described below, formulations containing such combinations are a matter of choice for those skilled in the art. Further, those skilled in the art will recognize that various coatings or other separation techniques may be used in cases where the combination of compounds are incompatible.

Compounds for use in accordance with the methods of the present invention can be provided in the form of pharmaceutically acceptable salts derived from inorganic or organic acids. Pharmaceutically acceptable salts are well-known in the art. For example, S. M. Berge et al. describe pharmaceutically acceptable salts in detail in J. Pharmaceutical Sciences, 66: 1 et seq. (1977). The salts can be prepared in situ during the final isolation and purification of the compounds or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

The at least one xanthine oxidoreductase inhibiting compound, urate lowering compound or salts thereof, may be formulated in a variety of ways that is largely a matter of choice depending upon the delivery route desired. For example, solid dosage forms for oral administration include capsules, tablets, pills, powders and granules. In such solid dosage forms, the xanthine oxidoreductase inhibiting compound or urate lowering compound may be mixed with at least one inert, pharmaceutically acceptable excipient or carrier, such as sodium citrate or dicalcium phosphate and/or a) fillers or extenders, such as, but not limited to, starches, lactose, sucrose, glucose, mannitol and silicic acid; b) binders, such as, but not limited to, carboxymethylcellulose, alginates, gelatin, polyvinylpyrrolidone, sucrose and acacia; c) humectants, such as, but not limited to glycerol; d) disintegrating agents, such as, but not limited to, agar-agar, calcium carbonate, potato or tapioca starch, alginic acid, certain silicates and sodium carbonate; e) solution retarding agents, such as, but not limited to, paraffin; f) absorption accelerators, such as, but not limited to, quaternary ammonium compounds; g) wetting agents, such as, but not limited to, cetyl alcohol and glycerol monostearate; h) absorbents, such as, but not limited to, kaolin and bentonite clay; and i) lubricants, such as, but not limited to, talc, calcium stearate, magnesium stearate, solid polyethylene glycols, sodium lauryl sulfate and mixtures thereof.

Solid compositions of a similar type may also be employed as fillers in soft and hard-filled gelatin capsules using such excipients as lactose or milk sugar as well as high molecular weight polyethylene glycols and the like.

The solid dosage forms of tablets, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions that can be used include polymeric substances and waxes.

Liquid dosage forms for oral administration include pharmaceutically acceptable emulsions, solutions, suspensions, syrups and elixirs. In addition to the xanthine oxidoreductase inhibiting compounds, the liquid dosage forms may contain inert diluents commonly used in the art such as, for example, water or other solvents, solubilizing agents and emulsifiers, such as, but not limited to, ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

The compositions can also be delivered through a catheter for local delivery at a target site, via an intracoronary stent (a tubular device composed of a fine wire mesh), or via a biodegradable polymer.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include, but are not limited to, water, ethanol, polyols (propylene glycol, polyethylene glycol, glycerol, and the like), vegetable oils (such as olive oil), injectable organic esters such as ethyl oleate, and suitable mixtures thereof.

These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form can be brought about by the use of agents delaying absorption, for example, aluminum monostearate and gelatin.

Suspensions, in addition to the active compounds (i.e., xanthine oxidoreductase inhibiting compounds, urate lowering compounds or salts thereof), may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Proper fluidity can be maintained, for example, by the use of coating materials such as lecithin, by the maintenance of the required particle size in the case of dispersions and by the use of surfactants.

In some cases, in order to prolong the effect of the drug (i.e. xanthine oxidoreductase inhibiting compounds, urate lowering compounds or salts thereof), it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Dosage forms for topical administration of the compounds of this present invention include powders, sprays, ointments and inhalants. The active compound(s) is mixed under sterile conditions with a pharmaceutically acceptable carrier and any needed preservatives, buffers or propellants, which can be required. Ophthalmic formulations, eye ointments, powders and solutions are also contemplated as being within the scope of this invention.

It will be understood that formulations used in accordance with the present invention generally will comprise a therapeutically effective amount of one or more xanthine oxidoreductase inhibiting compounds, one or more urate lowering compounds or combinations thereof.

Formulations of the present invention are administered and dosed in accordance with sound medical practice, taking into account the clinical condition of the individual patient, the site and method of administration, scheduling of administration, and other factors known to medical practitioners.

Therapeutically effective or prophylactically effective amounts for purposes herein thus can readily be determined by such considerations as are known to those skilled in the art. The daily therapeutically effective or prophylactically effective amount of the xanthine oxidoreductase inhibiting compounds administered to a patient in single or divided doses range from about 0.01 to about 750 milligram per kilogram of body weight per day (mg/kg/day). More specifically, a patient may be administered from about 5.0 mg to about 300 mg once daily, preferably from about 20 mg to about 240 mg once daily and most preferably from about 40 mg to about 120 mg once daily of xanthine oxidoreductase inhibiting compounds. Of course, it will be understood by one skilled in the art that other dosage regimens may be utilized, such as dosing more than once per day, utilizing extended, controlled, or modified release dosage forms, and the like in order to achieve the desired result of preventing the progression of a subject's joint damage or preventing a subject's joint damage.

The daily therapeutically effective or prophylactically effective amount of the at least one urate lowering compound administered to a patient in single or divided doses range from about 0.01 to about 900 milligram per kilogram of body weight per day (mg/kg/day). More specifically, a patient may be administered from about 25 mg to about 600 mg once daily, preferably from about 50 mg to about 300 mg once daily of a urate lowering compound. Of course, it will be understood by one skilled in the art that other dosage regimens may be utilized, such as dosing more than once per day, utilizing extended, controlled, or modified release dosage forms, and the like in order to achieve the desired result of preventing the progression of a subject's joint damage or preventing a subject's joint damage.

By way of example, and not of limitation, examples of the present invention will now be given.

### Example 1

### A Double-Blind, Multicenter, Placebo-Controlled Study Designed to Evaluate the effect of 2-[3-cyano-4-(2-methylpropoxy)phenyl]-4-methylthiazole-5-carboxylic Acid on Joint Damage in Hyperuricemic Subjects with Early Gout Study Design

This is a phase 2, multi-center, double-blind, randomized, placebo-controlled, study to evaluate the efficacy of 2-[3-cyano-4-(2-methylpropoxy)phenyl]-4-methylthiazole-5-carboxylic acid on joint damage in hyperuricemic subjects with early gout.

More specifically, as will be discussed in more detail below, the purpose of this study is to evaluate the 2-[3-cyano-4-(2-methylpropoxy)phenyl]-4-methylthiazole-5-carboxylic acid compared to placebo on joint damage in hyperuricemic subjects with early gout who are urate lowering therapy (ULT) naïve with a history of a single acute flare located within right or left metatarsophalangeal (MTP), ankle, metacarpophalangeal (MCP), interphalangeal (IP), proximal interphalangeal (PIP), or distal interphalangeal (DIP) joint.

Subjects will make an initial visit to the clinic for Screening (Day −21). Subjects that meet the American Rheumatism Association (ARA) diagnostic criteria for gout, with the exception of having experienced only one gout flare, and that have a sUA level ≧7.0 mg/dL will be screened for the study. All subjects will receive gout flare prophylaxis for the first 6 months of the study. Subjects should not undergo X-radiation imaging (X-ray), Magnetic Resonance Imaging (MRI) or Dual Energy Computed Tomography (DECT) procedures at time of Screening unless the sUA inclusion criterion has been met. Subjects previously on ULT will be excluded. The joint with the history of a single acute gout flare within the right or left MTP, ankle, MCP, IP, PIP, or DIP joint will be identified at Screening and followed with imaging throughout the study. Subjects will be scheduled for both X-rays and MRI of the single affected and contralateral joint. Additionally, some pre-identified sites will perform DECT of both hands (full) or both feet (full) with the affected joint and the contralateral joint identified at Screening.

Subjects randomized to 2-[3-cyano-4-(2-methylpropoxy)phenyl]-4-methylthiazole-5-carboxylic acid will initially receive the 40 mg dose QD. Subjects will return to the site on Day 14 (+5 days) for sUA assessment. If their sUA is 6.0 mg/dL at Day 14, they will receive 2-[3-cyano-4-(2-methylpropoxy)phenyl]-4-methylthiazole-5-carboxylic acid 80 mg QD at Month 1 (+5 days), and will remain on this dose for the remainder of the study. Subjects with sUA <6.0 mg/dL at Day 14 (+5 days) will remain on 2-[3-cyano-4-(2-methylpropoxy)phenyl]-4-methylthiazole-5-carboxylic acid 40 mg QD throughout the study. Subjects randomized to placebo will have no dose adjustments.

Following Study Day 1, subjects will return to the clinic for study visits at Day 15 (+5 days), Month 1 (+5 days), Month 6 (±7 days), and every 6 months (±7 days) until Final Visit on Month 24 or the Early Termination Visit. Clinical assessments and laboratory safety tests will be conducted during these visits. X-rays of the affected joint and the contralateral joint will be performed every 6 months (Month 6, Month 12, Month 18, Month 24 or the Early Termination Visit). MRI with contrast of both the affected joint and the contralateral joint will be performed at the Month 12, and Month 24 or the Early Termination Visit (±30 days). Measurements of sUA will be conducted at Screening, Day 14, Month 1, Month 6, Month 12, Month 18, Month 24 or Early Termination Visit. Subjects will be advised to withhold study medication on scheduled visit days. If study medication is not withheld, the visit should not be rescheduled and it will not be considered a protocol violation; however the taking of the study medication must be recorded in the source document. Subjects will be contacted throughout the study after the Day 1 visit by telephone to assess adverse events, compliance, concomitant medications and gout flares.

### Subject Population:

Males aged 18 years or older and females aged 45 years, with hyperuricemia (serum uric acid 7.0 mg/dL at Screening) and a history of only a single (one) gout flare prior to Screening.

### Number of Subjects:

Per treatment group: 200; Estimated total: 400

### Number of Sites:

Approximately 65 sites in the United States

### Dose Level(s):

2-[3-cyano-4-(2-methylpropoxy)phenyl]-4-methylthiazole-5-carboxylic acid 40 mg QD

2-[3-cyano-4-(2-methylpropoxy)phenyl]-4-methylthiazole-5-carboxylic acid will be increased to 80 mg QD at Month 1 if sUA is ≧6.0 mg/dL at Day 14

Placebo QD

### Route of Administration:

Oral

### Duration of Treatment:

24 Months

### Period of Evaluation:

21 day Screening Period

24 Month Double-Blind Treatment period

30 days post last dose spontaneous AE reporting period

### Primary Analysis:

The primary analysis will be the effect of 2-[3-cyano-4-(2-methylpropoxy)phenyl]-4-methylthiazole-5-carboxylic acid versus placebo at Month 24 on joint damage assessed based on modified SHARP Erosion Score since erosion appears earlier in gout. The effect on joint damage will also be assessed via modified SHARP joint space narrowing (JSN) score and the modified total (erosion plus JSN) SHARP score.

The secondary endpoints will include assessments of joint damage using the MRI OMERACT RAMRIS Scoring system. In the subset of subjects who have DECT performed, the changes in the modified SHARP erosion and RAMRIS scores will be summarized by the presence or absence of urate crystals in an exploratory analysis. Percentage of subjects with sUA <6.0 mg/dL at Month 24 will also be assessed.

### Main Criteria for Inclusion:

1. The subject has a history or presence of gout defined as having one or more of the following conditions of the American Rheumatism Association (ARA) criteria for the diagnosis of gout:
   A tophus proven to contain urate crystals by chemical or polarized light microscopic means and/or
   Characteristic urate crystals in the joint fluid and/or
   History of at least 6 of the following clinical, laboratory and X-ray phenomena*:
      maximum inflammation developed within 1 day
      monoarticular arthritis
      redness observed over joints
      first metatarsophalangeal joint painful or swollen
      unilateral first metatarsophalangeal joint attack
      unilateral tarsal joint attack
      tophus (proven or suspected)
      hyperuricemia
      asymmetric swelling within a joint on X-ray
      sub-cortical cysts without erosions on X-ray
      joint fluid culture negative for organisms during attacks
   *More than one flare criteria has been excluded for the purpose of the study
2. The subject is male and at least 18 years of age OR; Female ≧45 years of age and at least 2 years post-menopausal AND has a Follicle Stimulating Hormone (FSH) level ≧40 IU/L OR Female receiving hormone replacement therapy (HRT) must be ≧55 years of age (FSH level not required).
3. The subject has hyperuricemia defined as sUA level 7.0 mg/dL at Screening.
4. The subject has a history of (one) single acute gout flare located within right or left MTP, ankle, MCP, IP, PIP, or DIP joints prior to Screening.
5. The subject is capable of understanding and complying with protocol requirements.

### Main Criteria for Exclusion:

1. The subject was previously on ULT.
2. The subject has secondary hyperuricemia (e.g. due to myeloproliferative disorder or organ transplant).
3. The subject has a history of xanthinuria.
4. The subject has a known hypersensitivity to any component of the 2-[3-cyano-4-(2-methylpropoxy)phenyl]-4-methylthiazole-5-carboxylic acid formulation.
5. The subject has rheumatoid arthritis.
6. The subject has active peptic ulcer disease.
7. The subject has a history of cancer, except basal cell carcinoma of the skin, which has not been in remission for at least 5 years prior to the first dose of study medication.
8. The subject has experienced either a myocardial infarction (MI) or stroke within 90 days prior to the Screening visit.
9. The subject has ALT and/or AST values >2.0 the upper limit of normal during the Screening period.
10. The subject has a significant medical condition and/or conditions that, would interfere with the treatment, safety or compliance with the protocol at the discretion of the Investigator.
11. The subject has a history of drug abuse (defined as any illicit drug use) or a history of alcohol abuse within 5 years prior to the Screening visit. The subject consumes >14 alcoholic beverages/week.
12. The subject received any investigational medicinal product within 30 days prior to the Screening.
13. The subject has an estimated creatinine clearance of <60 mL/min, where creatinine clearance is calculated using the Cockcroft and Gault formula based on the Ideal Body Weight (IBW), as provided below:

$$Estimated creatinine clearance = \frac{( 140 - age ( year ) \times IBW ( kg ) ( female multiply by 0.85 )}{72 \times ( serum creatinine mg \text{/} dL )}$$

14. The subject has a serum creatinine at Screening >2.0 mg/dL.
15. The subject has a known history of infection with hepatitis B, hepatitis C, or human immunodeficiency virus.
16. The subject is a study site employee, or is an immediate family member (i.e. spouse, parent, child, and sibling) of a study site employee involved in conduct of this study.
17. The subject is unable to understand verbal or written English or any other language for which a certified translation of the approved informed consent is available.
18. The subject is required to take an excluded medication
   Urate lowering drug other than study medication
   Salicylates (chronic use of aspirin ≦325 mg day is allowed)
   Mercaptopurine
   Azathioprine
   Theophylline
   IV colchicines
   Pyrazinamide
   Sulfamethoxazole and trimethoprim
   Macrolides or ketolides, only when a subject is receiving concomitant colchicines
   Clopidogrel (only if subject is receiving concomitant naproxen)
19. Magnetic Resonance Imaging (MRI)—Exclusion Criteria
   The subject has a known hypersensitivity to gadolinium.
   The subject has a history of severe asthma.
   The subject has an electronically, magnetically, or mechanically activated implanted device.
   The subject contains any object that could present a potential hazard or interfere with MRI interpretation secondary to the artifact (i.e. metallic foreign bodies).
   The subject has a significant medical condition considered by the Investigator (or radiologist) to interfere with the subject's ability to receive gadolinium (e.g. Sickle cell anemia).
   The subject is ineligible for MRI based on the MR Procedure Screening Form.

### Main Criteria for Evaluation and Analyses:

Unless specified otherwise all endpoint evaluation and analyses will be based on the single affected joint.

The primary outcome (endpoint) measured for this study is mean change from baseline to Month 24 in the modified SHARP Erosion Score (mSES).

The key secondary outcomes (endpoints) measured are:
Mean change from baseline to Month 24 in RAMRIS score.
Mean change from baseline to Month 24 in the modified Total SHARP Score.
Mean change from baseline to Month 24 in the modified SHARP joint space narrowing Score.
Percentage of subjects with sUA <6.0 mg/dL at Month 24.

Other secondary outcomes (endpoints) measured for this study are:
Mean change from baseline to Month 6, Month 12, and Month 18 in the modified Total SHARP Score.
Mean change from baseline to Month 6 and Month 12 in the RAMRIS score.
Mean change from baseline to Month 6, Month 12, and Month 18 in the modified SHARP Erosion Score.
Mean change from baseline to Month 6, Month 12, and Month 18 in the modified SHARP joint space narrowing Score.
The mean number of flares requiring treatment from the end of the first year of treatment to the end of the study.
The mean change from baseline to Month 12 and Month 24 in the modified SHARP Erosion Score by presence or absence of urate crystals based on DECT findings (for the subset of subjects with DECT performed).
The mean change from baseline to Month 12 and Month 24 in the RAMRIS score by presence or absence of urate crystals based on DECT findings (for the subset of subjects with DECT performed).
The mean change from baseline to Month 24 in the modified SHARP erosion composite scores from full and foot radiographs.
The mean change from baseline to Month 24 in the modified SHARP joint space narrowing composite scores from full hand and foot radiographs.
The mean change from baseline to Month 24 in the modified total SHARP composite scores from full hand and foot radiographs

All of the imaging related endpoints based on the single affected joint will also be evaluated for the contralateral joint as exploratory analysis. The following safety variables will be used to describe the safety and tolerability of 2-[3-cyano-4-(2-methylpropoxy)phenyl]-4-methylthiazole-5-carboxylic acid, vital sign recordings, clinical laboratory test results, adverse event (AE) monitoring, and electrocardiogram (ECG) assessments.

### Sample Size Justification:

A total of 400 subjects (200 per treatment group) are planned to be enrolled into this study. The sample size will provide at least 90% power to detect a difference in the mean change from baseline to Month 24 of the modified SHARP Erosion Score (mSES) in the affected joint at a two-sided significance level of 0.05 assuming a mean change of 2.0 in the placebo group, 0.5 in the 2-[3-cyano-4-(2-methylpropoxy)phenyl]-4-methylthiazole-5-carboxylic acid group, and a standard deviation of 4.0 using the Wilcoxon-Mann-Whitney rank-sum statistic.

### Statistical Considerations:

### Efficacy Analysis

All efficacy and safety analyses will be analyzed using the Full Analysis Set (FAS). The FAS will consist of all subjects who were randomized and received at least one dose of double-blind study medication. Efficacy analyses will be analyzed according to the randomized treatment and safety analyses will be analyzed according to the actual treatment received.

Unless otherwise specified, all statistical tests and confidence intervals will be two-sided and conducted at the 0.05 significance level.

The mean change from baseline to Month 24 of the modified SHARP Erosion Score (mSES) will be compared between the placebo and 2-[3-cyano-4-(2-methylpropoxy)phenyl]-4-methylthiazole-5-carboxylic acid treatment groups using a Wilcoxon-Mann-Whitney Rank-Sum Test. Similarly, the mean change from baseline to Month 6, Month 12, and Month 24 of the RAMRIS score, to Month 6, Month 12, Month 18, and Month 24 of the modified Total SHARP Score (mTSS), to Month 6, Month 12 Month 18, and Month 24 of the modified SHARP joint spacing narrowing Score, and to Month 6, Month 12, and Month 18 in the mSES will be compared between the placebo and 2-[3-cyano-4-(2-methylpropoxy)phenyl]-4-methylthiazole-5-carboxylic acid treatment groups using a Wilcoxon-Mann-Whitney Rank-Sum Test. In addition for the subset of subjects with DECT performed, the mean change from baseline to Month 12 and to Month 24 in the mSES and in the RAMRIS score will be compared between the placebo and 2-[3-cyano-4-(2-methylpropoxy)phenyl]-4-methylthiazole-5-carboxylic acid treatment groups using a stratified Wilcoxon-Mann-Whitney Rank-Sum Test using presence and absence of urate crystals as determined by DECT as stratification variables. For the composite scores from full hand and foot radiographs, the mean change from baseline to Month 24 of the mSES, the mTSS, and the modified sharp joint space narrowing Score will be compared between the placebo and 2-[3-cyano-4-(2-methylpropoxy)phenyl]-4-methylthiazole-5-carboxylic acid treatment groups using the Wilcoxon-Mann-Whitney Rank-Sum Test.

Analysis of the percentage of subjects with sUA <6.0 mg/dL at Month 24 will be compared between the treatment groups using Fisher's exact test.

The flare rate from the end of the first year of treatment to the end of study with 95% confidence intervals will be summarized by treatment groups using the Negative Binomial (NB) model with treatment as a factor. Flare rate will be calculated as the number of flares from the first year of treatment to the end of study divided by the length of time on treatment after the first year.

Missing data in the primary analysis will be imputed with baseline score if subjects drop-out from the study due to any adverse reaction otherwise the last available post-baseline observation will be imputed. A sensitivity analysis will be conducted by assigning the median changes to all patients with missing values.

All efficacy analyses will be performed based on image reading scoring data of all visit images that will be read at the end of the study.

### Safety Analysis

Safety evaluation will include assessment of adverse events (AEs), laboratory tests, vital signs, physical examination findings, and electrocardiograms (ECGs).

Treatment-emergent AEs will be summarized using the safety analysis set. AEs will be summarized using the Medical Dictionary for Regulatory Agencies (MedDRA) coding dictionary. In general, AEs will be tabulated at each of the following levels: overall summary (subject with at least one AE), the MedDRA system organ class (SOC), the MedDRA high level term (HLT), and the MedDRA preferred term (PT). Tabulations at each level of a term will present count (n) and percentage of subjects reporting any event for that term. Subjects reporting more than one occurrence for the term (level) being summarized will only be counted once.

In addition, the number and percentage of subjects with adjudicated major adverse cardiovascular events will be summarized by treatment group. Events that occur no later than 30 days after the last dose of study medication will be included in this analysis.

What is claimed is:

1. A method of preventing the progression of joint damage in a subject, the method comprising the steps of:
selecting a patient having at least hyperuricemia and gout; and
administering to the subject a therapeutically effective amount of at least one compound, wherein said at least one compound is a xanthine oxidoreductase inhibitor or a pharmaceutically acceptable salt thereof, wherein the administration of said at least one compound to the subject prevents the progression of joint damage in said subject.

2. The method of claim 1, wherein the xanthine oxidoreductase inhibitor is selected from the group consisting of: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-cyano-4-(2,2-dimethylpropoxy)phenyl]-1H-pyrazole-4-carboxylic acid, pyrazolo[1,5-a]-1,3,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole and a pharmaceutically acceptable salt thereof.

3. The method of claim 2, wherein the compound is 2-[3-cyano-4-(2-methylpropoxy)phenyl]-4-methylthiazole-5-carboxylic acid or a pharmaceutically acceptable salt thereof.

4. The method of claim 2, wherein the compound is 2-[3-cyano-4-(3-hydroxy-2-methylpropoxy)phenyl]-4-methyl-5-thiazolecarboxylic acid or a pharmaceutically acceptable salt thereof.

5. The method of claim 2, wherein the compound is 2-[3-cyano-4-(2-hydroxy-2-methylpropoxy)phenyl]-4-methyl-5-thiazolecarboxylic acid or a pharmaceutically acceptable salt thereof.

6. The method of claim 1, wherein the subject has acute gouty arthritis, chronic gouty joint disease, tophaceous gout, or uric acid urolithiasis.

7. The method of claim 1, wherein the subject further exhibits at least one of: inflammation developed within one (1) day, monoarticular arthritis, redness observed over one or more joints, a first metatarsophalangeal joint painful or swollen, an unilateral first metatarsophalangeal joint attack, unilateral tarsal joint attack, tophus (proven or suspected), asymmetric swelling within a joint on X-ray, sub-cortical cysts without erosions, joint fluid culture negative for organisms during attacks, a tophus proven to contain urate crystals, characteristic urate crystals in the joint fluid or combinations thereof.

8. The method of claim 1, further comprising the step of assessing the subject's response to administration of the compound through radiographic imaging.

9. The method of claim 8, wherein the radiographic imaging is Magnetic Resonance Imaging (MRI), X-ray, or Dual Energy Computed Tomography (DECT).

10. A method of preventing joint damage in a subject, the method comprising the steps of:
selecting a patient having at least hyperuricemia and gout; and
administering to the subject a therapeutically effective amount of at least one compound, wherein said at least one compound is a xanthine oxidoreductase inhibitor or a pharmaceutically acceptable salt thereof, wherein the administration of said at least one compound to the subject prevents the progression of joint damage in said subject.

11. The method of claim 10, wherein the xanthine oxidoreductase inhibitor is selected from the group consisting of: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-cyano-4-(2,2-dimethylpropoxy)phenyl]-1H-pyrazole-4-carboxylic acid, pyrazolo[1,5-a]-1,3,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole and a pharmaceutically acceptable salt thereof.

12. The method of claim 11, wherein the compound is 2-[3-cyano-4-(2-methylpropoxy)phenyl]-4-methylthiazole-5-carboxylic acid or a pharmaceutically acceptable salt thereof.

13. The method of claim 11, wherein the compound is 2-[3-cyano-4-(3-hydroxy-2-methylpropoxy)phenyl]-4-methyl-5-thiazolecarboxylic acid or a pharmaceutically acceptable salt thereof.

14. The method of claim 11, wherein the compound is 2-[3-cyano-4-(2-hydroxy-2-methylpropoxy)phenyl]-4-methyl-5-thiazolecarboxylic acid or a pharmaceutically acceptable salt thereof.

15. The method of claim 10, wherein the subject has acute gouty arthritis, chronic gouty joint disease, tophaceous gout, or uric acid urolithiasis.

16. The method of claim 10, wherein the subject further exhibits at least one of: inflammation developed within one (1) day, monoarticular arthritis, redness observed over one or more joints, a first metatarsophalangeal joint painful or swollen, an unilateral first metatarsophalangeal joint attack, unilateral tarsal joint attack, tophus (proven or suspected), asymmetric swelling within a joint on X-ray, sub-cortical cysts without erosions, joint fluid culture negative for organisms during attacks, a tophus proven to contain urate crystals, characteristic urate crystals in the joint fluid or combinations thereof.

17. The method of claim 10, further comprising the step of assessing the subject's response to administration of the compound through radiographic imaging.

18. The method of claim 17, wherein the radiographic imaging is Magnetic Resonance Imaging (MRI), X-ray, or Dual Energy Computed Tomography (DECT).

19. A method of preventing the progression of joint damage in a subject the method comprising the steps of:
selecting a patient having at least hyperuricemia and gout; and administering to the subject a therapeutically effective amount of at least one compound, wherein said at least one compound comprises the formula: 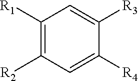
wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;
wherein R₃ and R₄ are each independently a hydrogen or A, B, C or D as shown below: 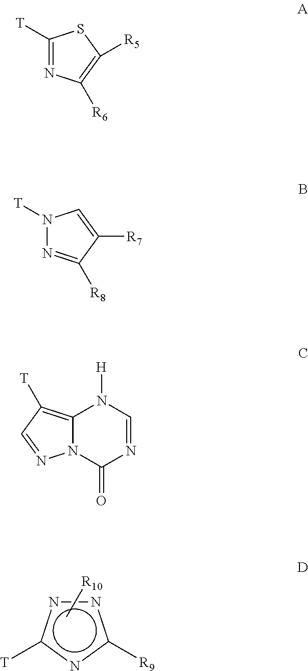
wherein T connects A, B, C or D to the aromatic ring shown above at R₁, R₂, R₃ or R₄,
wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₉ is an unsubstituted pyridyl group or a substituted pyridyl group; and
wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above, wherein the administration of said at least one compound to the subject prevents the progression of joint damage in said subject.

20. The method of claim 19, wherein the compound is 2-[3-cyano-4-(2-methylpropoxy)phenyl]-4-methylthiazole-5-carboxylic acid or a pharmaceutically acceptable salt thereof.

21. The method of claim 19, wherein the compound is 2-[3-cyano-4-(3-hydroxy-2-methylpropoxy)phenyl]-4-methyl-5-thiazolecarboxylic acid or a pharmaceutically acceptable salt thereof.

22. The method of claim 19, wherein the compound is 2-[3-cyano-4-(2-hydroxy-2-methylpropoxy)phenyl]-4-methyl-5-thiazolecarboxylic acid or a pharmaceutically acceptable salt thereof.

23. The method of claim 19, wherein the compound is 2-(3-cyano-4-hydroxyphenyl)-4-methyl-5-thiazolecarboxylic acid or a pharmaceutically acceptable salt thereof.

24. The method of claim 19, wherein the compound is 2-[4-(2-carboxypropoxy)-3-cyanophenyl]-4-methyl-5-thiazolecarboxylic acid or a pharmaceutically acceptable salt thereof.

25. The method of claim 19, wherein the compound is 1-3-cyano-4-(2,2-dimethylpropoxy)phenyl]-1H-pyrazole-4-carboxylic acid or a pharmaceutically acceptable salt thereof.

26. The method of claim 19, wherein the compound is pyrazolo[1,5-a]-1,3,5-triazin-4-(1H)-one, 8-[3-methoxy-4-(phenylsulfinyl)phenyl]-sodium salt (±).

27. The method of claim 19, wherein the compound is 3-(2-methyl-4-pyridyl)-5-cyano-4-isobutoxyphenyl)-1,2,4-triazole or a pharmaceutically acceptable salt thereof.

28. The method of claim 19, wherein the subject has acute gouty arthritis, chronic gouty joint disease, tophaceous gout, or uric acid urolithiasis.

29. The method of claim 19, wherein the subject further exhibits at least one of: inflammation developed within one (1) day, monoarticular arthritis, redness observed over one or more joints, a first metatarsophalangeal joint painful or swollen, an unilateral first metatarsophalangeal joint attack, unilateral tarsal joint attack, tophus (proven or suspected), asymmetric swelling within a joint on X-ray, sub-cortical cysts without erosions, joint fluid culture negative for organisms during attacks, a tophus proven to contain urate crystals, characteristic urate crystals in the joint fluid or combinations thereof.

30. The method of claim 19, further comprising the step of assessing the subject's response to administration of the compound through radiographic imaging.

31. The method of claim 30, wherein the radiographic imaging is Magnetic Resonance Imaging (MRI), X-ray, or Dual Energy Computed Tomography (DECT).

32. A method of preventing the progression of joint damage in a subject, the method comprising the steps of:
selecting a patient having at least hyperuricemia and gout; and
administering to the subject a therapeutically effective amount of at least one compound, wherein said at least one compound comprises the formula: 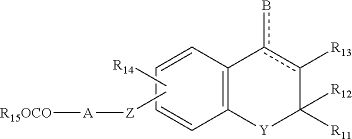
wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;
wherein R₁₃ is a hydrogen or a substituted or unsubstituted lower alkyl group;
wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R_{17′}, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R_{17′} are each independently a hydrogen or a substituted or unsubstituted lower alkyl;
wherein R₁₅ is a hydrogen or a pharmaceutically active ester-forming group;
wherein A is a straight or branched hydrocarbon radical having one to five carbon atoms;
wherein B is a halogen, an oxygen, or an ethylenedithio;
wherein Y is an oxygen, a sulfur, a nitrogen or a substituted nitrogen;
wherein Z is an oxygen, a nitrogen or a substituted nitrogen; and
the dotted line refers to either a single bond, a double bond, or two single bonds, wherein the administration of said at least one compound to the subject prevents the progression of joint damage in said subject.

33. A method of preventing joint damage in a subject, the method comprising the steps of:
selecting a patient having at least hyperuricemia and gout; and
administering to the subject a therapeutically effective amount of at least one compound, wherein said at least one compound comprises the formula: 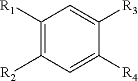
wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;
wherein R₃ and R₄ are each independently a hydrogen or A, B, C or D as shown below: 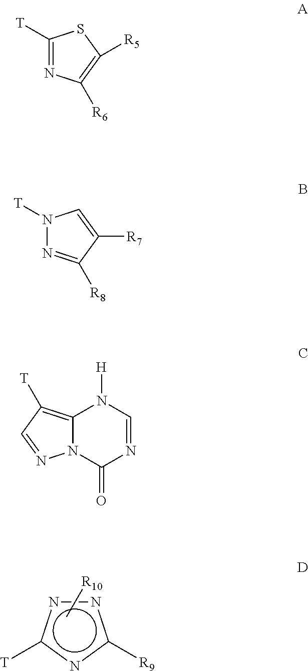
wherein T connects A, B, C or D to the aromatic ring shown above at R₁, R₂, R₃ or R₄,
wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₉ is an unsubstituted pyridyl group or a substituted pyridyl group; and
wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above, wherein the administration of said at least one compound to the subject prevents the progression of joint damage in said subject.

34. The method of claim 33, wherein the compound is 2-[3-cyano-4-(2-methylpropoxy)phenyl]-4-methylthiazole-5-carboxylic acid or a pharmaceutically acceptable salt thereof.

35. The method of claim 33, wherein the compound is 2-[3-cyano-4-(3-hydroxy-2-methylpropoxy)phenyl]-4-methyl-5-thiazolecarboxylic acid or a pharmaceutically acceptable salt thereof.

36. The method of claim 33, wherein the compound is 2-[3-cyano-4-(2-hydroxy-2-methylpropoxy)phenyl]-4-methyl-5-thiazolecarboxylic acid or a pharmaceutically acceptable salt thereof.

37. The method of claim 33, wherein the compound is 2-(3-cyano-4-hydroxyphenyl)-4-methyl-5-thiazolecarboxylic acid or a pharmaceutically acceptable salt thereof.

38. The method of claim 33, wherein the compound is 2-[4-(2-carboxypropoxy)-3-cyanophenyl]-4-methyl-5-thiazolecarboxylic acid or a pharmaceutically acceptable salt thereof.

39. The method of claim 33, wherein the compound is 1-3-cyano-4-(2,2-dimethylpropoxy)phenyl]-1H-pyrazole-4-carboxylic acid or a pharmaceutically acceptable salt thereof.

40. The method of claim 33, wherein the compound is pyrazolo[1,5-a]-1,3,5-triazin-4-(1H)-one, 8-[3-methoxy-4-(phenylsulfinyl)phenyl]-sodium salt (±).

41. The method of claim 33, wherein the compound is 3-(2-methyl-4-pyridyl)-5-cyano-4-isobutoxyphenyl)-1,2,4-triazole or a pharmaceutically acceptable salt thereof.

42. The method of claim 33, wherein the subject has acute gouty arthritis, chronic gouty joint disease, tophaceous gout, or uric acid urolithiasis.

43. The method of claim 33, wherein the subject further exhibits at least one of: inflammation developed within one (1) day, monoarticular arthritis, redness observed over one or more joints, a first metatarsophalangeal joint painful or swollen, an unilateral first metatarsophalangeal joint attack, unilateral tarsal joint attack, tophus (proven or suspected), asymmetric swelling within a joint on X-ray, sub-cortical cysts without erosions, joint fluid culture negative for organisms during attacks, a tophus proven to contain urate crystals, characteristic urate crystals in the joint fluid or combinations thereof.

44. The method of claim 33, further comprising the step of assessing the subject's response to administration of the compound through radiographic imaging.

45. The method of claim 44, wherein the radiographic imaging is Magnetic Resonance Imaging (MRI), X-ray, or Dual Energy Computed Tomography (DECT).

46. A method of preventing joint damage in a subject, the method comprising the steps of:
selecting a patient having at least hyperuricemia and gout; and
administering to the subject a therapeutically effective amount of at least one compound, wherein said at least one compound comprises the formula: 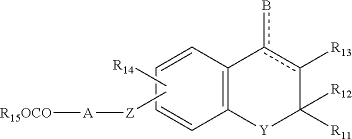
wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;
wherein R₁₃ is a hydrogen or a substituted or unsubstituted lower alkyl group;
wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R_{17′}, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R_{17′} are each independently a hydrogen or a substituted or unsubstituted lower alkyl;
wherein R₁₅ is a hydrogen or a pharmaceutically active ester-forming group;
wherein A is a straight or branched hydrocarbon radical having one to five carbon atoms;
wherein B is a halogen, an oxygen, or an ethylenedithio;
wherein Y is an oxygen, a sulfur, a nitrogen or a substituted nitrogen;
wherein Z is an oxygen, a nitrogen or a substituted nitrogen; and
the dotted line refers to either a single bond, a double bond, or two single bonds, wherein the administration of said at least one compound to the subject prevents the progression of joint damage in said subject.

47. A method of identifying a patient suitable for treatment with at least one xanthine oxidoreductase inhibitor in order to prevent joint damage or the progression of joint damage in a subject, the method comprising the step of:
obtaining a test sample from a subject;
determining whether said subject is hyperuricemic and has gout; wherein if said subject is hyperurecimeic and has gout identifying said patient as eligible for treatment with at least one xanthine oxidoreductase inhibitor in order to prevent joint damage or the progression of joint damage in said subject.

48. The method of claim 47, wherein the method further comprising determining if the subject further exhibits at least one of: inflammation developed within one (1) day, monoarticular arthritis, redness observed over one or more joints, a first metatarsophalangeal joint painful or swollen, an unilateral first metatarsophalangeal joint attack, unilateral tarsal joint attack, tophus (proven or suspected), asymmetric swelling within a joint on X-ray, sub-cortical cysts without erosions, joint fluid culture negative for organisms during attacks, a tophus proven to contain urate crystals, characteristic urate crystals in the joint fluid or combinations thereof.

49. A method of preventing the progression of joint damage in a subject, the method comprising the steps of:
selecting a patient having at least hyperuricemia and early gout; and
administering to the subject a therapeutically effective amount of at least one compound, wherein said at least one compound is a xanthine oxidoreductase inhibitor or a pharmaceutically acceptable salt thereof, wherein the administration of said at least one compound to the subject prevents the progression of joint damage in said subject.

50. The method of claim 49, wherein the xanthine oxidoreductase inhibitor is selected from the group consisting of: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-cyano-4-(2,2-dimethylpropoxy)phenyl]-1H-pyrazole-4-carboxylic acid, pyrazolo[1,5-a]-1,3,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole and a pharmaceutically acceptable salt thereof.

51. The method of claim 50, wherein the compound is 2-[3-cyano-4-(2-methylpropoxy)phenyl]-4-methylthiazole-5-carboxylic acid or a pharmaceutically acceptable salt thereof.

52. The method of claim 50, wherein the compound is 2-[3-cyano-4-(3-hydroxy-2-methylpropoxy)phenyl]-4-methyl-5-thiazolecarboxylic acid or a pharmaceutically acceptable salt thereof.

53. The method of claim 50, wherein the compound is 2-[3-cyano-4-(2-hydroxy-2-methylpropoxy)phenyl]-4-methyl-5-thiazolecarboxylic acid or a pharmaceutically acceptable salt thereof.

54. The method of claim 49, wherein the subject has acute gouty arthritis, chronic gouty joint disease, tophaceous gout, or uric acid urolithiasis.

55. The method of claim 49, wherein the subject further exhibits at least one of: inflammation developed within one (1) day, monoarticular arthritis, redness observed over one or more joints, a first metatarsophalangeal joint painful or swollen, an unilateral first metatarsophalangeal joint attack, unilateral tarsal joint attack, tophus (proven or suspected), asymmetric swelling within a joint on X-ray, sub-cortical cysts without erosions, joint fluid culture negative for organisms during attacks, a tophus proven to contain urate crystals, characteristic urate crystals in the joint fluid or combinations thereof.

56. The method of claim 49, further comprising the step of assessing the subject's response to administration of the compound through radiographic imaging.

57. The method of claim 56, wherein the radiographic imaging is Magnetic Resonance Imaging (MRI), X-ray, or Dual Energy Computed Tomography (DECT).

58. A method of preventing joint damage in a subject, the method comprising the steps of:
selecting a patient having at least hyperuricemia and early gout; and
administering to the subject a therapeutically effective amount of at least one compound, wherein said at least one compound is a xanthine oxidoreductase inhibitor or a pharmaceutically acceptable salt thereof, wherein the administration of said at least one compound to the subject prevents the progression of joint damage in said subject.

59. The method of claim 58, wherein the xanthine oxidoreductase inhibitor is selected from the group consisting of: 2-[3-cyano-4-(2-methylpropoxy)phenyl]-4-methylthiazole-5-carboxylic acid, 2-[3-cyano-4-(3-hydroxy-2-methylpropoxy)phenyl]-4-methyl-5-thiazolecarboxylic acid, 2-[3-cyano-4-(2-hydroxy-2-methylpropoxy)phenyl]-4-methyl-5-thiazolecarboxylic acid, 2-(3-cyano-4-hydroxyphenyl)-4-methyl-5-thiazolecarboxylic acid, 2-[4-(2-carboxypropoxy)-3-cyanophenyl]-4-methyl-5-thiazolecarboxylic acid, 1-(3-cyano-4-(2,2-dimethylpropoxy)phenyl)-1H-pyrazole-4-carboxylic acid, 1-3-cyano-4-(2,2-dimethylpropoxy)phenyl]-1H-pyrazole-4-carboxylic acid, pyrazolo[1,5-a]-1,3,5-triazin-4-(1H)-one, 8-[3-methoxy-4-(phenylsulfinyl)phenyl]-sodium salt (±), 3-(2-methyl-4-pyridyl)-5-cyano-4-isobutoxyphenyl)-1,2,4-triazole and a pharmaceutically acceptable salt thereof.

60. The method of claim 59, wherein the compound is 2-[3-cyano-4-(2-methylpropoxy)phenyl]-4-methylthiazole-5-carboxylic acid or a pharmaceutically acceptable salt thereof.

61. The method of claim 59, wherein the compound is 2-[3-cyano-4-(3-hydroxy-2-methylpropoxy)phenyl]-4-methyl-5-thiazolecarboxylic acid or a pharmaceutically acceptable salt thereof.

62. The method of claim 59, wherein the compound is 2-[3-cyano-4-(2-hydroxy-2-methylpropoxy)phenyl]-4-methyl-5-thiazolecarboxylic acid or a pharmaceutically acceptable salt thereof.

63. The method of claim 58, wherein the subject has acute gouty arthritis, chronic gouty joint disease, tophaceous gout, or uric acid urolithiasis.

64. The method of claim 58, wherein the subject further exhibits at least one of: inflammation developed within one (1) day, monoarticular arthritis, redness observed over one or more joints, a first metatarsophalangeal joint painful or swollen, an unilateral first metatarsophalangeal joint attack, unilateral tarsal joint attack, tophus (proven or suspected), asymmetric swelling within a joint on X-ray, sub-cortical cysts without erosions, joint fluid culture negative for organisms during attacks, a tophus proven to contain urate crystals, characteristic urate crystals in the joint fluid or combinations thereof.

65. The method of claim 58, further comprising the step of assessing the subject's response to administration of the compound through radiographic imaging.

66. The method of claim 65, wherein the radiographic imaging is Magnetic Resonance Imaging (MRI), X-ray, or Dual Energy Computed Tomography (DECT).

67. A method of preventing the progression of joint damage in a subject the method comprising the steps of:
selecting a patient having at least hyperuricemia and early gout; and administering to the subject a therapeutically effective amount of at least one compound, wherein said at least one compound comprises the formula: 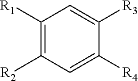
wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;
wherein R₃ and R₄ are each independently a hydrogen or A, B, C or D as shown below: 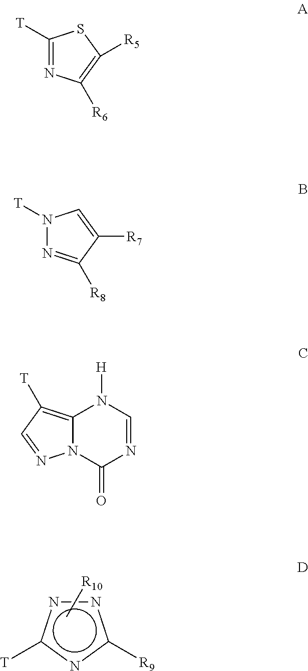
wherein T connects A, B, C or D to the aromatic ring shown above at R₁, R₂, R₃ or R₄,
wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₉ is an unsubstituted pyridyl group or a substituted pyridyl group; and
wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above, wherein the administration of said at least one compound to the subject prevents the progression of joint damage in said subject.

68. The method of claim 67, wherein the compound is 2-[3-cyano-4-(2-methylpropoxy)phenyl]-4-methylthiazole-5-carboxylic acid or a pharmaceutically acceptable salt thereof.

69. The method of claim 67, wherein the compound is 2-[3-cyano-4-(3-hydroxy-2-methylpropoxy)phenyl]-4-methyl-5-thiazolecarboxylic acid or a pharmaceutically acceptable salt thereof.

70. The method of claim 67, wherein the compound is 2-[3-cyano-4-(2-hydroxy-2-methylpropoxy)phenyl]-4-methyl-5-thiazolecarboxylic acid or a pharmaceutically acceptable salt thereof.

71. The method of claim 67, wherein the compound is 2-(3-cyano-4-hydroxyphenyl)-4-methyl-5-thiazolecarboxylic acid or a pharmaceutically acceptable salt thereof.

72. The method of claim 67, wherein the compound is 2-[4-(2-carboxypropoxy)-3-cyanophenyl]-4-methyl-5-thiazolecarboxylic acid or a pharmaceutically acceptable salt thereof.

73. The method of claim 67, wherein the compound is 1-3-cyano-4-(2,2-dimethylpropoxy)phenyl]-1H-pyrazole-4-carboxylic acid or a pharmaceutically acceptable salt thereof.

74. The method of claim 67, wherein the compound is pyrazolo[1,5-a]-1,3,5-triazin-4-(1H)-one, 8-[3-methoxy-4-(phenylsulfinyl)phenyl]-sodium salt (±).

75. The method of claim 67, wherein the compound is 3-(2-methyl-4-pyridyl)-5-cyano-4-isobutoxyphenyl)-1,2,4-triazole or a pharmaceutically acceptable salt thereof.

76. The method of claim 67, wherein the subject has acute gouty arthritis, chronic gouty joint disease, tophaceous gout, or uric acid urolithiasis.

77. The method of claim 67, wherein the subject further exhibits at least one of: inflammation developed within one (1) day, monoarticular arthritis, redness observed over one or more joints, a first metatarsophalangeal joint painful or swollen, an unilateral first metatarsophalangeal joint attack, unilateral tarsal joint attack, tophus (proven or suspected), asymmetric swelling within a joint on X-ray, sub-cortical cysts without erosions, joint fluid culture negative for organisms during attacks, a tophus proven to contain urate crystals, characteristic urate crystals in the joint fluid or combinations thereof.

78. The method of claim 67, further comprising the step of assessing the subject's response to administration of the compound through radiographic imaging.

79. The method of claim 78, wherein the radiographic imaging is Magnetic Resonance Imaging (MRI), X-ray, or Dual Energy Computed Tomography (DECT).

80. A method of preventing the progression of joint damage in a subject, the method comprising the steps of:
selecting a patient having at least hyperuricemia and early gout; and
administering to the subject a therapeutically effective amount of at least one compound, wherein said at least one compound comprises the formula: 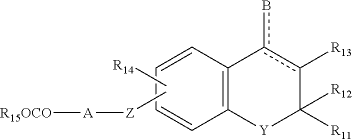
wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;
wherein R₁₃ is a hydrogen or a substituted or unsubstituted lower alkyl group;
wherein R₁₄ is one or two radicals selected from a group consisting of a hydrogen, a halogen, a nitro group, a substituted or unsubstituted lower alkyl, a substituted or unsubstituted phenyl, —OR₁₆ and SO₂NR₁₇R_{17′}, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R_{17′} are each independently a hydrogen or a substituted or unsubstituted lower alkyl;
wherein R₁₅ is a hydrogen or a pharmaceutically active ester-forming group;
wherein A is a straight or branched hydrocarbon radical having one to five carbon atoms;
wherein B is a halogen, an oxygen, or an ethylenedithio;
wherein Y is an oxygen, a sulfur, a nitrogen or a substituted nitrogen;
wherein Z is an oxygen, a nitrogen or a substituted nitrogen; and
the dotted line refers to either a single bond, a double bond, or two single bonds, wherein the administration of said at least one compound to the subject prevents the progression of joint damage in said subject.

81. A method of preventing joint damage in a subject, the method comprising the steps of:
selecting a patient having at least hyperuricemia and early gout; and
administering to the subject a therapeutically effective amount of at least one compound, wherein said at least one compound comprises the formula: 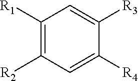
wherein R₁ and R₂ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, a phenylsulfinyl group or a cyano (—CN) group;
wherein R₃ and R₄ are each independently a hydrogen or A, B, C or D as shown below: 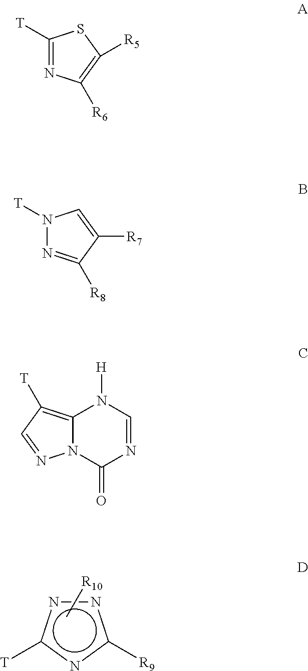
wherein T connects A, B, C or D to the aromatic ring shown above at R₁, R₂, R₃ or R₄,
wherein R₅ and R₆ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₇ and R₈ are each independently a hydrogen, a hydroxyl group, a COOH group, an unsubstituted or substituted C₁-C₁₀ alkyl group, an unsubstituted or substituted C₁-C₁₀ alkoxy, an unsubstituted or substituted hydroxyalkoxy, COO-Glucoronide or COO-Sulfate;
wherein R₉ is an unsubstituted pyridyl group or a substituted pyridyl group; and
wherein R₁₀ is a hydrogen or a lower alkyl group, a lower alkyl group substituted with a pivaloyloxy group and in each case, R₁₀ bonds to one of the nitrogen atoms in the 1,2,4-triazole ring shown above, wherein the administration of said at least one compound to the subject prevents the progression of joint damage in said subject.

82. The method of claim 81, wherein the compound is 2-[3-cyano-4-(2-methylpropoxy)phenyl]-4-methylthiazole-5-carboxylic acid or a pharmaceutically acceptable salt thereof.

83. The method of claim 81, wherein the compound is 2-[3-cyano-4-(3-hydroxy-2-methylpropoxy)phenyl]-4-methyl-5-thiazolecarboxylic acid or a pharmaceutically acceptable salt thereof.

84. The method of claim 81, wherein the compound is 2-[3-cyano-4-(2-hydroxy-2-methylpropoxy)phenyl]-4-methyl-5-thiazolecarboxylic acid or a pharmaceutically acceptable salt thereof.

85. The method of claim 81, wherein the compound is 2-(3-cyano-4-hydroxyphenyl)-4-methyl-5-thiazolecarboxylic acid or a pharmaceutically acceptable salt thereof.

86. The method of claim 81, wherein the compound is 2-[4-(2-carboxypropoxy)-3-cyanophenyl]-4-methyl-5-thiazolecarboxylic acid or a pharmaceutically acceptable salt thereof.

87. The method of claim 81, wherein the compound is 1-3-cyano-4-(2,2-dimethylpropoxy)phenyl]-1H-pyrazole-4-carboxylic acid or a pharmaceutically acceptable salt thereof.

88. The method of claim 81, wherein the compound is pyrazolo[1,5-a]-1,3,5-triazin-4-(1H)-one, 8-[3-methoxy-4-(phenylsulfinyl)phenyl]-sodium salt (±).

89. The method of claim 81, wherein the compound is 3-(2-methyl-4-pyridyl)-5-cyano-4-isobutoxyphenyl)-1,2,4-triazole or a pharmaceutically acceptable salt thereof.

90. The method of claim 81, wherein the subject has acute gouty arthritis, chronic gouty joint disease, tophaceous gout, or uric acid urolithiasis.

91. The method of claim 81, wherein the subject further exhibits at least one of: inflammation developed within one (1) day, monoarticular arthritis, redness observed over one or more joints, a first metatarsophalangeal joint painful or swollen, an unilateral first metatarsophalangeal joint attack, unilateral tarsal joint attack, tophus (proven or suspected), asymmetric swelling within a joint on X-ray, sub-cortical cysts without erosions, joint fluid culture negative for organisms during attacks, a tophus proven to contain urate crystals, characteristic urate crystals in the joint fluid or combinations thereof.

92. The method of claim 81, further comprising the step of assessing the subject's response to administration of the compound through radiographic imaging.

93. The method of claim 92, wherein the radiographic imaging is Magnetic Resonance Imaging (MRI), X-ray, or Dual Energy Computed Tomography (DECT).

94. A method of preventing joint damage in a subject, the method comprising the steps of:
selecting a patient having at least hyperuricemia and early gout; and
administering to the subject a therapeutically effective amount of at least one compound, wherein said at least one compound comprises the formula: 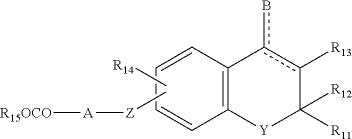
wherein R₁₁ and R₁₂ are each independently a hydrogen, a substituted or unsubstituted lower alkyl group, a substituted or unsubstituted phenyl, or R₁₁ and R₁₂ may together form a four- to eight-membered carbon ring together with the carbon atom to which they are attached;
wherein R₁₃ is a hydrogen or a substituted or unsubstituted lower alkyl group;
wherein R₁₄ is one or two radicals selected from a group consisting of a hydrogen, a halogen, a nitro group, a substituted or unsubstituted lower alkyl, a substituted or unsubstituted phenyl, —OR₁₆ and —SO₂NR₁₇R_{17′}, wherein R₁₆ is a hydrogen, a substituted or unsubstituted lower alkyl, a phenyl-substituted lower alkyl, a carboxymethyl or ester thereof, a hydroxyethyl or ether thereof, or an allyl; R₁₇ and R_{17′} are each independently a hydrogen or a substituted or unsubstituted lower alkyl;
wherein R₁₅ is a hydrogen or a pharmaceutically active ester-forming group;
wherein A is a straight or branched hydrocarbon radical having one to five carbon atoms;
wherein B is a halogen, an oxygen, or an ethylenedithio;
wherein Y is an oxygen, a sulfur, a nitrogen or a substituted nitrogen;
wherein Z is an oxygen, a nitrogen or a substituted nitrogen; and
the dotted line refers to either a single bond, a double bond, or two single bonds, wherein the administration of said at least one compound to the subject prevents the progression of joint damage in said subject.
